(19) United States
(12) Patent Application Publication
(10) Pub. No.: US 2026/0294369 A1
(43) Pub. Date: Oct. 1, 2026

(54) SAFETY STRUCTURE FOR CEILING MOUNTED MEDICAL IMAGING SYSTEM AND METHOD OF INSTALLING SAME

(71) Applicant: GE Precision Healthcare LLC, Waukesha, WI (US)

(72) Inventors: Xiaoyi Guo, Beijing (CN); Guoli Pan, Beijing (CN); Julien Marcotte, Saint Vrain (FR); Romain Chatelin, Plaisir (FR); Weidong Lv, Beijing (CN); Jianwei Liu, Beijing (CN)

(21) Appl. No.: 19/097,261

(22) Filed: Apr. 1, 2025

(51) Int. Cl. A61B 6/00; A61B 6/10

(52) U.S. Cl. CPC A61B 6/4464; A61B 6/10

(57) ABSTRACT

A safety hook of a ceiling mount assembly operable to receive a load of a medical imaging system in a failure condition of the ceiling mount assembly is provided. The safety hook includes a main body, a hook, and locking pin. The main body includes a first end slidably insertable in an interior channel of a main beam of a carriage frame of the ceiling mount assembly. The hook is configured to partially surround a protrusion of a ceiling rail. The main body includes first and second pin holes. The locking pin is inserted through an aperture in the main beam of the carriage frame and the first pin hole to secure the safety hook in a retracted position, and is inserted through the aperture in the main beam of the carriage frame and the second pin hole to secure the safety hook in an extended position.

### FIELD

Certain embodiments relate to ceiling mounts for medical imaging systems. More specifically, certain embodiments relate to a safety structure for a ceiling mounted medical imaging system that includes safety hooks to prevent the ceiling mounted medical imaging system from falling if a screw or other attachment mechanism of the ceiling mount assembly fails. Various embodiments relate to methods of installing a medical imaging system with a ceiling mount having safety hooks for preventing the medical imaging system from falling if a screw or other attachment mechanism of the ceiling mount assembly fails.

### BACKGROUND

Medical diagnostic imaging systems generate images of a patient through exposure to an energy source, such as X-rays passing through a patient, for example. The generated images may be used to provide a medical diagnosis of a patient, among other things. In some cases, the medical diagnostic imaging system may be mounted to a ceiling in an examination room. The medical diagnostic imaging system may be movable and rotatable to acquire medical image data from different positions and/or angles. One example of a ceiling mounted medical diagnostic imaging system is C-arm X-ray diagnostic equipment. The term C-arm generally refers to an X-ray imaging device having a rigid and/or articulating structural member having an X-ray source and an image detector assembly that are each located at an opposing end of the structural member so that the X-ray source and the image detector face each other. The structural member is typically “C” shaped and so is referred to as a C-arm. In this manner, X-rays emitted from the X-ray source can impinge on the image detector and provide an X-ray image of the object or objects that are placed between the X-ray source and the image detector. The weight of a C-arm X-ray diagnostic medical imaging system may be 800 kilograms or more. If a screw or other attachments mechanism of the mounting assembly were to loosen or break and the C-arm X-ray diagnostic medical imaging system were to fall on a patient, the result could be catastrophic.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

Ceiling mount assemblies and methods of installing the ceiling mount assemblies are provided, wherein the ceiling mount assemblies have a safety structure including safety hooks for preventing a ceiling mounted medical imaging system from falling if a screw or other attachment mechanism of the ceiling mount assembly fails, substantially as shown in and/or described in connection with at least one of the figures, as set forth more completely in the claims.

These and other advantages, aspects, and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram illustrating components of an exemplary X-ray imaging system, in accordance with various embodiments.

FIG. 2 is a schematic diagram of a side view of an exemplary X-ray imaging system mounted to a ceiling, in accordance with various embodiments.

FIG. 3 is a schematic diagram of a lateral view of an X-ray imaging system mounted to a ceiling by a lower ceiling mount and an upper ceiling mount, in accordance with various embodiments.

FIG. 4 is a schematic diagram of a perspective view of an exemplary carriage frame of a chassis of an upper ceiling mount slidably coupled to ceiling rails of a rail system, in accordance with various embodiments.

FIG. 5 is a schematic diagram of a perspective view of exemplary carriage safety hooks of a carriage frame of a chassis of an upper ceiling mount positioned to surround protrusions of ceiling rails of a rail system, in accordance with various embodiments.

FIG. 6 is a schematic diagram of a top view of exemplary carriage safety hooks of a carriage frame of a chassis of an upper ceiling mount positioned to surround protrusions of ceiling rails of a rail system, in accordance with various embodiments.

FIG. 7 is a schematic diagram of a top perspective view of exemplary carriage safety hooks of a portion of a carriage frame of a chassis of an upper ceiling mount positioned to surround protrusions of ceiling rails of a rail system, in accordance with various embodiments.

FIG. 8 is a schematic diagram of a side perspective view of exemplary carriage safety hooks of a portion of a carriage frame of a chassis of an upper ceiling mount positioned to surround protrusions of ceiling rails of a rail system, in accordance with various embodiments.

FIG. 9 is a schematic diagram of a lateral view of an exemplary carriage safety hook extending from a main beam of a carriage frame and positioned to surround a protrusion of a ceiling rail, in accordance with various embodiments.

FIG. 10 is a schematic diagram of a cross-sectional view of an exemplary carriage safety hook extending from a main beam of a carriage frame and positioned to surround a protrusion of a ceiling rail, in accordance with various embodiments.

FIG. 11 is a schematic diagram of a perspective view of an exemplary carriage safety hook with locking pin, in accordance with various embodiments.

FIG. 12 is a schematic diagram of a lateral view of a main beam of a carriage frame of a chassis having carriage safety hooks provided in a retracted position, in accordance with various embodiments.

FIG. 13 is a schematic diagram of a lateral view of a main beam of a carriage frame of a chassis having carriage safety hooks provided in an extended position, in accordance with various embodiments.

FIG. 14 is a schematic diagram of a lateral view of an exemplary carriage frame of a chassis of an upper ceiling mount pivotally coupled to a lower ceiling mount by a swivel bearing, the carriage frame having carriage safety hooks provided in a retracted position prior to installation on protrusions of ceiling rails, in accordance with various embodiments.

FIG. 15 is a schematic diagram of a lateral view of an exemplary carriage frame of a chassis of an upper ceiling mount pivotally coupled to a lower ceiling mount by a swivel bearing, the carriage frame having carriage safety hooks provided in an extended position after installation on protrusions of ceiling rails, in accordance with various embodiments.

FIG. 16 is a schematic diagram of lateral views of a preferred installation method of a medical imaging system to ceiling rails of a rail system by a ceiling mount assembly having carriage safety hooks, in accordance with various embodiments.

FIG. 17 is a schematic diagram of lateral views of an alternative installation method of a medical imaging system to ceiling rails of a rail system by a ceiling mount assembly having carriage safety hooks, in accordance with various embodiments.

### DETAILED DESCRIPTION

Certain embodiments may be found in ceiling mount assemblies and methods of installing the ceiling mount assemblies, wherein the ceiling mount assemblies have a safety structure including safety hooks for preventing a ceiling mounted medical imaging system from falling if attachment mechanisms of the ceiling mount assembly fail. For example, aspects of the present disclosure have the technical effect of ensuring the safety of patients and operators by substantially reducing and/or eliminating the risk of a ceiling mounted medical imaging system falling if attachment mechanisms of the mounting assembly fail. In certain embodiments, a ceiling mount assembly comprises an upper ceiling mount and a lower ceiling mount. The upper ceiling mount comprises a chassis having a carriage frame slidably coupled to protrusions of ceiling rails of a rail assembly by slidable connectors, such as linear guide assemblies having rollers. The carriage frame comprises carriage safety hooks extending from main beams of the carriage frame and operable to partially surround the protrusions of the ceiling rails. In various embodiments, the carriage safety hooks of the carriage frame of the upper ceiling mount take no load during normal operation. Instead, the carriage safety hooks carry the load of the medical imaging system only if a failure of attachment mechanisms occur, such as a failure of one or more of the slidable connectors (e.g., linear guide assemblies). In an exemplary embodiment, the carriage safety hooks are extendable from and retractable into the main beams such that the upper ceiling mount of the ceiling mount assembly may be lifted vertically to install the medical imaging system to the ceiling rails of the rail system instead of having to slide the upper ceiling mount horizontally on to the ceiling rails from an end of the ceiling rails, thereby allowing installation of the medical imaging system to ceilings of smaller examination rooms and/or allowing longer ceiling rails for greater lateral movement within an examination room.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random-access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical, and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image.

Furthermore, the term processor or processing unit, as used herein, refers to any type of processing unit that can carry out the required calculations needed for the various embodiments, such as single or multi-core Central Processing Unit (CPU), Accelerated Processing Unit (APU), Graphic Processing Unit (GPU), Digital Signal Processor (DSP), Field Programmable Gate Array (FPGA), System on a Chip (SoC), Application-Specific Integrated Circuit (ASIC), or a combination thereof.

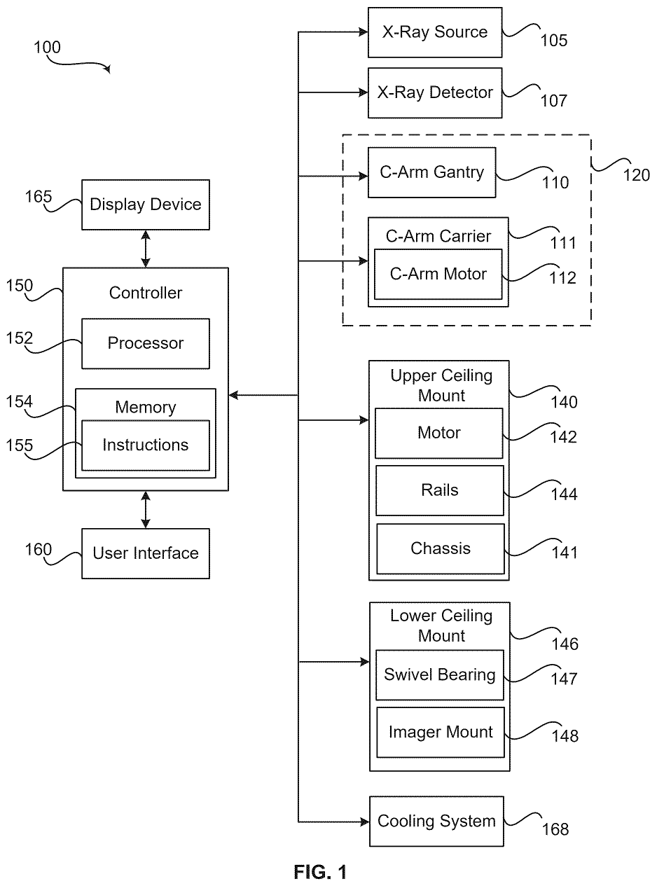

FIG. 1 is a block diagram illustrating components of an exemplary X-ray imaging system 100, in accordance with various embodiments. Referring to FIG. 1, there is shown an X-ray imaging system 100 that comprises a controller 150, a user interface 160, a display device 165, an X-ray source 105, and X-ray detector 107, a gantry structure 120, an upper ceiling mount 140, a lower ceiling mount 146, and a cooling system 168, among other things. In certain embodiments, the X-ray imaging system 100 is configured to perform vascular imaging, such as digital subtraction angiography (DSA). The X-ray imaging system 100 is configured to be mounted to a ceiling. In various embodiments, the X-ray imaging system 100 is mounted to the ceiling at a fixed location. In certain embodiments, the X-ray imaging system 100 is mounted to the ceiling but may move along the ceiling (e.g., via chassis that moves along a rail system). The X-ray imaging system 100 includes an X-ray source 105 (or X-ray radiation source) and an X-ray detector 107 mounted on a C-arm gantry 110 (e.g., C-arm). In an exemplary embodiment, the C-arm gantry 110 may be made of carbon fiber.

The C-arm gantry 110 is part of a gantry structure 120. The gantry structure 120 includes a C-arm motor 112 for adjusting the position of the C-arm gantry 110. More specifically, the C-arm gantry 110 is mechanically coupled to a C-arm carrier 111 (e.g., C-arm rotation device) which includes the C-arm motor 112, and the C-arm motor 112 may be driven to adjust the position of the C-arm gantry 110 with respect to the C-arm carrier 111. For example, the C-arm carrier 111 in conjunction with the C-arm motor 112 is configured to rotate the C-arm gantry 110 in an orbital direction relative to the C-arm carrier 111. In certain embodiments, the C-arm carrier 111 (via a motorized system) is configured to rotate a pivot (e.g., pivot point) where the C-arm carrier 111 is coupled to an end of an arm (e.g., L-arm) coupled or mounted to the ceiling. The C-arm carrier 111 rotates about a rotational axis (e.g., horizontal axis) of the pivot. In representative embodiments having an L-arm, the L-arm may rotate about a location where the other end of the L-arm (i.e., the end of the L-arm not connected to the pivot) is coupled to or mounted to the ceiling (via a lower ceiling mount 146). In various embodiments, the C-arm carrier 111 is coupled to or mounted to the ceiling (via the lower ceiling mount 146).

The upper ceiling mount 140 is coupled to a ceiling. In certain embodiments, the C-arm carrier 111 is coupled to the upper ceiling mount 140 via an L-arm (e.g., via the end of the L-arm not connected to the pivot) coupled to the lower ceiling mount 146. The upper ceiling mount 140 is configured to move (e.g., translocate) the X-ray imaging system 100 from one location to another location (e.g., in a linear direction) on the ceiling. The upper ceiling mount 140 includes a chassis 141 having a carriage frame. The upper ceiling mount 140 also includes a motor 142 and a rail system 144 (e.g., having rails). The rail system 144 is directly coupled to the ceiling. The motor 142 is configured to drive movement of the chassis 141 and, thus, the X-ray imaging system 100 along the rail system 144 (e.g., to adjust a position of the X-ray imaging system 100). Accordingly, the carriage frame off the chassis 141 may be slidably mounted by slidable connectors to the ceiling rails of the rail system 144. In various embodiments, the slidable connectors may be linear guide assemblies comprising rollers for slidably mounting the chassis 141 to the ceiling rails. In an exemplary embodiment, carriage safety hooks are provided on the carriage frame of the chassis 141 to prevent the chassis 141 from falling from the ceiling rails of the rail system 144 if the slidable connectors fail, as discussed in more detail below.

The lower ceiling mount 146 is configured to rotatably couple the gantry structure 120 to the upper ceiling mount 140. In various embodiment, the lower ceiling mount 146 may be operable to dampen vibrations that occur when the C-arm gantry 110 (e.g., made of carbon fiber) moves and/or deaccelerates (e.g., during the deacceleration phase) to a stop during movement of the gantry structure 120 along multiple axes or directions (e.g., combined directions). For example, the movement of the gantry structure 120 may include the rotational movement of the C-arm gantry 110 in the orbital direction, rotational movement about the axis of the pivot where the C-arm carrier 111 is coupled to an end of an arm (e.g., L-arm), and/or rotation about where an axis of where the lower ceiling mount 146 (e.g., axis of the swivel bearing 147) is coupled the ceiling. The lower ceiling mount 146 is configured to dampen vibrations caused by movement of the gantry structure 120 for an entirety of the gantry structure 120 along the multiple directions. The multiple directions may include orthogonal directions. For example, the orthogonal directions may include a vertical direction extending between a floor and the ceiling and a horizontal direction that is both perpendicular to the vertical direction and parallel with the floor.

The lower ceiling mount 146 includes a swivel bearing 147 and an imager mount 148. The imager mount 148 includes a body having a first end and a second end opposite the first end, a first mount connection disposed on the first end, and a second mount connection coupled to the second end. The first mount connection and the second mount connection both directly contact both the gantry structure 120 and the body. In an exemplary embodiment, the first mount connection includes one or more mounting brackets, damping pads or dampers (e.g., made of elastomeric material such as rubber), and mounting screws and/or any suitable attachment mechanism. In certain embodiments, the second mount connection includes a shaft and bushings coupled to opposite ends of the shaft, and wherein the shaft extends through a portion of the gantry structure 120.

The swivel bearing 147 pivotally couples the gantry structure 120 to the upper ceiling mount 140 (e.g., the chassis) when the X-ray imaging system 100 is mounted to the ceiling. The swivel bearing 147 is disposed on a side of the body facing the ceiling. In certain embodiments, the body is made of a material that is stiffer than both the first mount connection and the second mount connection. The swivel bearing 147 is configured to enable the gantry structure 120 to rotate 360 degrees about a rotational axis of the swivel bearing 147.

The X-ray imaging system 100 further includes a controller 150 comprising suitable logic, circuitry, interfaces, and/or code that may be operable to control operation of the X-ray imaging system 100. The controller 150 comprises a processor 152 and a non-transitory memory 154. A method for controlling the X-ray imaging system 100 may be stored as executable instructions 155 in the non-transitory memory 154 and executed by the processor 152.

The memory 154 may be one or more computer-readable memories integrated with the X-ray imaging system 100 and/or communicatively coupled (e.g., over a network) to the X-ray imaging system 100, such as a Picture Archiving and Communication System (PACS), a server, a hard disk, floppy disk, CD, CD-ROM, DVD, compact storage, flash memory, random access memory, read-only memory, electrically erasable and programmable read-only memory, optical storage medium, magnetic storage medium, solid-state storage medium, and/or any suitable memory. The memory 154 may include databases, libraries, sets of information, or other storage accessed by and/or incorporated with the processor 152, for example. The memory 154 may be able to store data temporarily or permanently, for example. The memory 154 may store processor-executable software code or instructions (e.g., firmware or software) 155, which are tangibly stored on a non-transitory computer readable medium. Additionally or alternatively, the memory 154 may store medical image data, data generated by the processor 152, and/or any suitable data.

The processor 152 may comprise suitable logic, circuitry, interfaces and/or code that may be operable to process medical image data for generating medical images (e.g., X-ray images) for presentation on a display device 165. The processor 152 is operable to perform one or more processing operations on the acquired medical image data. In an exemplary embodiment, the processor 152 may be operable to perform display processing and/or control processing, among other things. Acquired medical image data may be processed in real-time during an imaging examination as the medical image data is received. Additionally or alternatively, the medical image data may be stored temporarily during an imaging examination and processed in less than real-time in a live or off-line operation. In various embodiments, the processed image data can be presented at the display device 165 and/or may be stored at the memory 154. The memory 154 may be a local archive, a Picture Archiving and Communication System (PACS), or any suitable device for storing images and related information. The processor 152 may be one or more central processing units, graphic processing units, microprocessors, microcontrollers, and/or the like. The processor 152 may be an integrated component, or may be distributed across various locations, for example. In an exemplary embodiment, the processor 132 may be capable of receiving input information from a user interface 160 and/or memory 154, generating an output displayable by a display device 165, and manipulating the output in response to input information from a user interface 160, among other things. The processor 152 may be capable of executing any of the method(s) and/or set(s) of instructions 155 discussed herein in accordance with the various embodiments, for example.

The user interface 160 may be utilized to receive input from a user or operator of the X-ray imaging system 100. The user interface 160 may be communicatively coupled to the controller 150 for providing commands input by a user via the user interface 160 to the controller 150. For example, the user interface 160 may receive inputs of patient data, image acquisition parameters, settings, configuration parameters, select protocols and/or templates, and the like. In an exemplary embodiment, the user interface 160 may be operable to configure, manage and/or control operation of one or more components and/or modules in the X-ray imaging system 100. In this regard, the user interface 160 may be operable to configure, manage and/or control operation of the controller 150, user interface 160, display device 165, X-ray source 105, X-ray detector 107, C-arm gantry 110, C-arm carrier 111, upper ceiling mount 140, lower ceiling mount 146 and/or the cooling system 168. The user interface 160 may include hard button(s), soft button(s), rotary encoder(s), a touchscreen, motion tracking, voice recognition, a mousing device, keyboard, trackball, joystick(s), a touchpad, camera, and/or any other device capable of receiving a user directive. In certain embodiments, one or more of the user interfaces 160 may be integrated into other components, such as the display device 165, for example. As an example, user interface 160 may include a touchscreen display. In some examples the user interface 160 may be remotely located relative to the X-ray imaging system 100. For example, the user interface 160 may be communicatively coupled to the controller 150 and/or the X-ray imaging system 100 via a wired or wireless connection.

As an example, a user of the X-ray imaging system 100 may input a desired isocenter position via the user interface 160. The controller 150 may then determine position adjustments to one or more of the C-arm gantry 110 and/or the upper ceiling mount 140 to align an isocenter of the X-ray imaging system 100 with the desired isocenter position. As another example, a user of the X-ray imaging system 100 may directly control the position of one or more components of the X-ray imaging system 100 relative to other components of the X-ray imaging system 100 via the user interface 160. For example, the user may directly input, via a joystick or knob, for example, position adjustments to one or more components of the X-ray imaging system 100. As another example, the motion of the components of the X-ray imaging system 100 may be pre-programmed such that the user does not directly control any movement, but instead initiates the start of the pre-programmed motion. The motion may include complex motions, with continuous motion of the isocenter.

The controller 150 is further communicatively coupled to a display device 165 for displaying one or more X-ray images acquired via the X-ray detector 107. Further, in some examples, one or more of the controller 150, the user interface 160, and the display device 165 may be positioned away from (e.g., remotely from) the remaining components of the X-ray imaging system 100. The display device 165 may be any device capable of communicating visual information to a user. For example, a display device 165 may include a liquid crystal display, a light emitting diode display, and/or any suitable display or displays.

The X-ray imaging system 100 may further include a cooling system 168 for cooling the X-ray source 105 and/or the X-ray detector 107. The cooling system 168 may include one or more flexible tubes and a pump, as an illustrative and non-limiting example, providing cooling fluid to the X-ray source 105 to transfer thermal energy away from the X-ray source 105. The cooling system 168 may actively cool the X-ray source 105 and the X-ray detector 107 independently, or in some examples may cool the X-ray detector 107 by any suitable type of derivation of the cooling circuit for the X-ray source 105.

Components of the X-ray imaging system 100 may be implemented in software, hardware, firmware, and/or the like. The various components of the X-ray imaging system 100 may be communicatively linked. Components of the X-ray imaging system 100 may be implemented separately and/or integrated in various forms. For example, the display device 165 and the user input device 130 may be integrated as a touchscreen display.

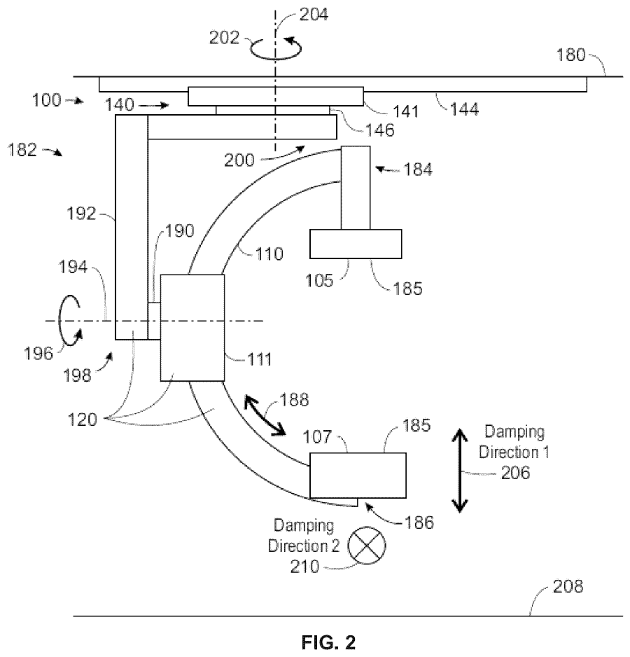

FIG. 2 is a schematic diagram of a side view of an exemplary X-ray imaging system 100 mounted to a ceiling 180, in accordance with various embodiments. The X-ray imaging system 100 of FIG. 2 shares various characteristics with the X-ray imaging system of FIG. 1 as described above. Referring to FIG. 2, the X-ray imaging system 100 is provided in an examination room 182 having a ceiling 180 and a floor 208. The X-ray imaging system 100 comprises an X-ray source 105, and X-ray detector 107, a gantry structure 120 having a C-arm gantry 110, a C-arm carrier 111 and a mounting structure 192, an upper ceiling mount 140, and a lower ceiling mount, among other things.

The gantry structure 120 comprises a C-arm gantry 110, a C-arm carrier 111, and a mounting structure 192. In certain embodiments, the C-arm gantry 110 is made of carbon fiber. The X-ray radiation source 105 is coupled to a first end 184 of the C-arm gantry 110 and the X-ray detector 107 is coupled to a second end 186 of the C-arm gantry 110 opposite the first end 184 (e.g., forming the image chain 185). The C-arm gantry 110 is coupled to the C-arm carrier 111 (e.g., C-arm rotation device), which is configured to rotate the C-arm gantry 110 in an orbital direction 188 relative to the C-arm carrier 111 about an isocenter of the X-ray radiation source 105 and the X-ray detector 107. The C-arm carrier 111 includes rollers (e.g., guiding rollers) to guide movement of the C-arm gantry 110 relative to the C-arm carrier 111.

The C-arm carrier 111 is coupled to a pivot 190 (e.g., pivot point or shaft). The pivot 190 is coupled to the mounting structure 192. The pivot 190 is driven by a motor (e.g., C-arm motor or any suitable motor) configured to rotate both the C-arm carrier 111 and the C-arm gantry 110 about a rotational axis 194 (e.g., horizontal axis) of the pivot 190 as indicated by arrow 196. In certain embodiments, the mounting structure 192 is an L-arm coupled to the upper ceiling mount 140 via the lower ceiling mount 146. The pivot 190 is coupled to a first end 198 of the L-arm 192 and the lower ceiling mount 146 is coupled to a second end 200 of the L-arm 192. The lower ceiling mount 146 is configured to couple the gantry structure 120 to the upper ceiling mount 140 (and, thus, mount the X-ray imaging system 100 to the ceiling 180). In certain embodiments, the L-arm may rotate about an end of the L-arm coupled to the upper ceiling mount 140 via the swivel bearing of the lower ceiling mount 146. In particular, the L-arm (and the gantry structure 120) rotate in direction 202 about a rotational axis 204 of the swivel bearing.

The C-arm carrier 111 is coupled to the upper ceiling mount 140 via the L-arm 192 (e.g., via the end 200 of the L-arm not connected to the pivot 190) coupled to the lower ceiling mount 146. The upper ceiling mount 140 is coupled to the ceiling 180. The upper ceiling mount 140 is configured to linearly translate the X-ray imaging system 100 from one location to another location across the ceiling 180. The upper ceiling mount 140 includes a chassis 141, a motor 142, and a rail system 144 (e.g., having rails). The rail system 144 is directly coupled to the ceiling 180. In various embodiments, the chassis 141 comprises a carriage frame slidably mounted by slidable connectors to the ceiling rails of the rail system 144. In certain embodiments, the slidable connectors may be linear guide assemblies comprising rollers for slidably mounting the carriage frame of the chassis 141 to the ceiling rails. In an exemplary embodiment, carriage safety hooks are provided on the carriage frame of the chassis 141 to prevent the chassis 141 from falling from the ceiling rails of the rail system 144 if the linear guide assemblies or any suitable slidable connectors fail, as discussed in more detail below. The motor 142 is configured to drive movement of the chassis 141 and, thus, the X-ray imaging system 100 along the rail system 144 (e.g., to adjust a position of the X-ray imaging system 100).

The lower ceiling mount 146 is coupled to the upper ceiling mount 140. In particular, a swivel bearing of the lower ceiling mount 146 is rotatably coupled to a chassis 141 of the upper ceiling mount 140. In various embodiments, the lower ceiling mount 146 is configured to dampen vibrations caused by motion of the gantry structure 120 for an entirety of the gantry structure 120. For example, the movement of the gantry structure 120 may include the rotational movement of the C-arm gantry 110 in the orbital direction 188, rotational movement in the direction 196 about the axis 194 of the pivot 190 where the C-arm carrier 111 is coupled to an end of the mounting structure 192 (e.g., L-arm), and/or rotational movement in the direction 202 about the 204 axis of where the lower ceiling mount 146 (e.g., axis 204 of the swivel bearing) is coupled to the ceiling 180. The lower ceiling mount 146 is configured to dampen vibrations caused by movement of the gantry structure 120 for an entirety of the gantry structure 120 along the multiple directions. The multiple directions may include orthogonal directions. For example, the orthogonal directions may include a vertical direction 206 extending between a floor 208 and the ceiling 180 and a horizontal direction 210 (into the page in FIG. 2) that is both perpendicular to the vertical direction 206 and parallel with the floor 208.

The lower ceiling mount includes a swivel bearing (e.g., which couples the gantry structure to the chassis 141 of the upper ceiling mount 140 installed on the ceiling 180) and an imager mount. The swivel bearing is disposed on a side of the body facing the ceiling 180. The imager mount includes a body having a first end and a second end opposite the first end, a first mount connection disposed on the first end, and a second mount connection coupled to the second end. The first mount connection and the second mount connection both directly contact both the mounting structure 192 of the gantry structure 120 and the body. In an exemplary embodiment, the first mount connection includes one or more mounting brackets, damping pads or dampers (e.g., made of elastomeric material such as rubber), and mounting screws and/or any suitable attachment mechanism. In certain embodiments, the second mount connection includes a shaft and bushings coupled to opposite ends of the shaft, and wherein the shaft extends through a portion of the gantry structure 120.

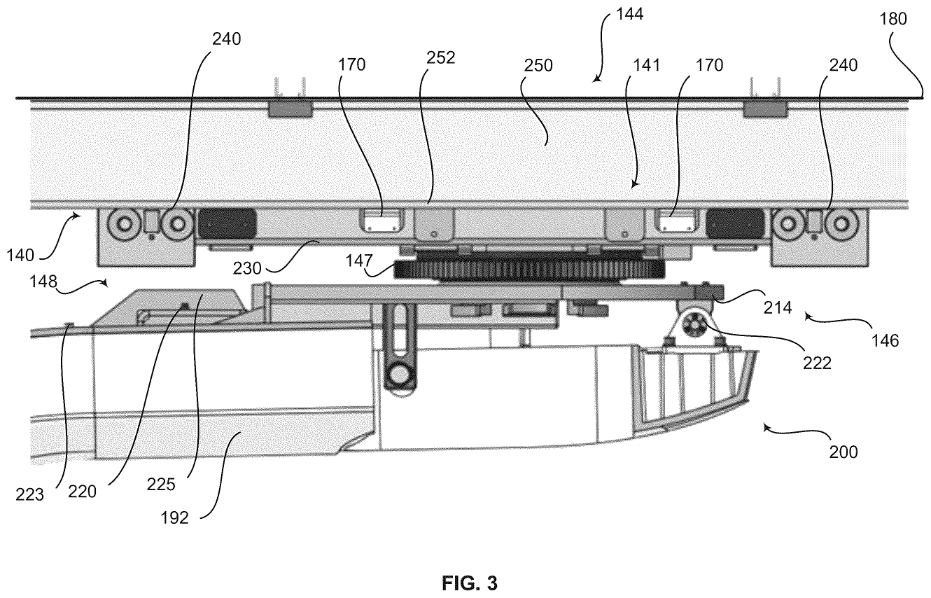

FIG. 3 is a schematic diagram of a lateral view of an X-ray imaging system 100 mounted to a ceiling 180 by a lower ceiling mount 146 and an upper ceiling mount 140, in accordance with various embodiments. The upper ceiling mount 140 and lower ceiling mount 146 for mounting a mounting structure 192 (e.g., L-arm) of a medical imaging system (e.g., an X-ray system 100) to a ceiling 180 as shown in FIG. 3 share various characteristics with the upper ceiling mount 140 and lower ceiling mount 146 for mounting a mounting structure 192 (e.g., L-arm) of a medical imaging system (e.g., an X-ray system 100) to a ceiling 180 described above with reference to FIGS. 1 and 2.

Referring to FIG. 3, a lower ceiling mount 146 is coupled to a mounting structure 192 of a medical imaging system, such as a gantry structure of an X-ray imaging system. Only a portion of the mounting structure 192 is shown in FIG. 3. In various embodiments, the portion of the mounting structure 192 may be an L-arm 192 of a gantry structure 120 as shown in FIGS. 1 and 2. The lower ceiling mount 146 is configured to be rotatably coupled to an upper ceiling mount 140 installed on the ceiling 180. The upper ceiling mount 140 is configured to rectilinearly move the medical imaging system (e.g., X-ray imaging system 100) from one location to another location across the ceiling 180. The upper ceiling mount 140 comprises a chassis 141, a rail system 144, and a motor. The rail system 144 comprises parallel rails 250 coupled to the ceiling 180. The parallel rails 250 comprise protrusions 252 extending horizontally from at least an interior side (i.e., the side facing the corresponding parallel rail 250) of each of the rails 250. The chassis 141 comprises a carriage frame 230 having slidable connectors, such as linear guide assemblies 240 with rollers that slidably couple the carriage frame 230 of the chassis 141 to the protrusions 252 extending from the interior of the rails 252 of the rail system 144. The carriage frame 230 of the chassis 141 comprises carriage safety hooks 170 positioned to surround the protrusion 252 of the ceiling rails 250. In various embodiments, in a normal condition when a proper connection is present between the carriage frame 230 of the chassis 141 and the protrusion 252 of the ceiling rails 250 by the linear guide assemblies 240 and/or any suitable slidable connectors, the carriage safety hooks 170 may not receive a load of the medical imaging system (e.g., X-ray imaging system 100) mounted to the ceiling via the lower 146 and upper 140 ceiling mounts. In certain embodiments, the carriage safety hooks 170 may not contact the protrusion 252 of the ceiling rails 250 in a normal condition. However, in a failure condition when one or more of the linear guide assemblies 240 or any suitable slidable connectors coupling the carriage frame 230 of the chassis 141 to the protrusion 252 of the ceiling rails 250 fail, the carriage safety hooks 170 are operable to receive (e.g., catch) the protrusion 252 of the ceiling rails 250 to prevent the carriage frame 230, and the medical imaging system coupled thereto, from falling from the ceiling rails 250, as discussed in more detail below. The motor is operable to drive movement of the chassis 141 linearly along the ceiling rails 250 to adjust a position of the medical imaging system (e.g., X-ray imaging system 100).

The lower ceiling mount 146 includes a swivel bearing 147 and an imager mount 148. A bottom side of the swivel bearing 147 is disposed on a top side 224 of a body 214 of the imager mount 148 facing the ceiling 180. A top side of the swivel bearing 147 may be rotatably coupled to the chassis 141 of the upper ceiling mount 140. For example, the swivel bearing 147 may comprise a fixed outer ring attached to the chassis 141 and a pivotable inner ring coupled to the imager mount 148. A motor may drive rotation of the inner ring of the swivel bearing 147 to adjust an orientation of the medical imaging system (e.g., X-ray imaging system 100) by rotating the imager mount 148 to a desired position. The swivel bearing 147 is configured to enable the medical imaging system (e.g., X-ray imaging system 100) to rotate 360 degrees in the direction 202 about the rotational axis 204 of the swivel bearing 147.

The imager mount 148 includes a body 214 having a first end 216 and a second end 218 opposite the first end 216, a first mount connection 220, 223, 225 disposed on the first end 216, and a second mount connection 222 coupled to the second end 218. As shown in FIG. 3, both the first mount connection 220, 223, 225 and the second mount connection 222 directly contact both the mounting structure 192 and the body 214. In certain embodiments, the first mount connection 220, 223, 225 includes a mounting bracket 225 attached at a first end to the body 214 by attachment mechanisms 220, such as one or more damping pads or dampers (e.g., made of an elastomeric material such as rubber) and mounting screws or any suitable attachment mechanisms. A second end of the mounting bracket 225 is attached to the mounting structure 192 by attachment mechanisms 223, such as mounting screws or any suitable attachment mechanisms. The first mount connection 220, 223, 225 is disposed on a top side 224 of the body 214 (e.g., facing the ceiling 180). In particular, the first mount connection 220, 223, 225 is disposed between and contacts the mounting structure 192 and the body 214. The second mount connection 222 includes a shaft and bushings coupled to opposite ends of the shaft. The shaft extends through a portion of a second end 200 of the mounting structure 192 and portions of the body 214 located on a bottom side (e.g., facing away from the ceiling 180) of the body 214. In various embodiments, the body 214 is made of a material that is stiffer than both the first mount connection 220, 223, 225 and the second mount connection 222. The stiffer body 214 minimizes the movement of the gantry structure 120 during damping of vibrations.

In various embodiments, the lower ceiling mount 146 is configured to dampen vibrations caused by motion of the medical imaging system (e.g., a gantry structure 120 of an X-ray imaging system 100) for an entirety of the medical imaging system along the multiple directions. The multiple directions may include orthogonal directions. For example, the orthogonal directions may include a vertical direction 206 (damping direction 1) extending between the floor and the ceiling 180 and the horizontal direction 210 (damping direction 2) that is both perpendicular to the vertical direction 206 and parallel with the floor 208 as depicted in FIG. 2. As depicted in FIG. 2, the damping directions 1 and 2 may include a circumferential element, such as about a rotational axis of the shaft and side to side movement of lateral sides of the lower ceiling mount 146, respectively.

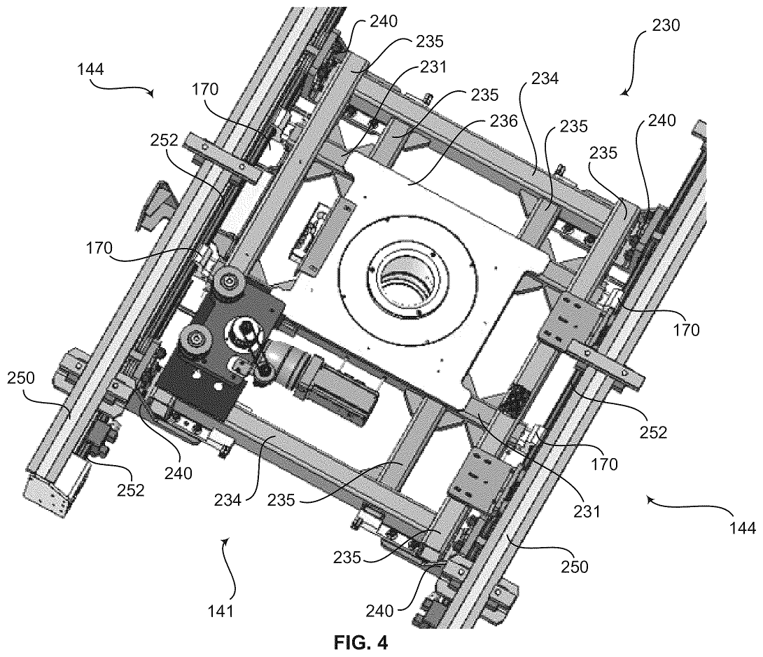

FIG. 4 is a schematic diagram of a perspective view of an exemplary carriage frame 230 of a chassis 141 of an upper ceiling mount 140 slidably coupled to ceiling rails 250 of a rail system 144, in accordance with various embodiments. The chassis 141 having the carriage frame 230 slidably mounted to ceiling rails 250 of rail system 144 by linear guide assemblies 240 as shown in FIG. 4 shares various characteristics with chassis 141 of the upper ceiling mount 140 slidably coupled to the rail system 144 as described above with reference to FIGS. 1 and 2. Referring to FIG. 4, a chassis 141 of an upper ceiling mount 140 comprises a carriage frame 230 slidably coupled to ceiling rails 250 of a rail system 144 by linear guide assemblies 240, or any suitable slidable connectors. The rail system 144 comprises parallel rails 250 that are configured to be mounted to a ceiling. The parallel rails 250 comprise protrusions 252 extending horizontally from at least an interior side (i.e., the side facing the corresponding parallel rail 250) of each of the rails 250. The chassis 141 comprises a carriage frame 230 having slidable connectors, such as linear guide assemblies 240 with rollers, that slidably couple the carriage frame 230 of the chassis 141 to the protrusions 252 extending from the interior of the rails 252 of the rail system 144. A motor is operable to drive movement of the chassis 141 linearly along the ceiling rails 250 to adjust a position of the medical imaging system (e.g., X-ray imaging system 100).

In various embodiments, the carriage frame 230 of the chassis 141 comprises lateral beams 231, 234 and longitudinal beams 235. The lateral beams 231, 234 include two outer lateral beams 234 and two interior main lateral beams 231. The longitudinal beams 235 include two outer longitudinal beams 235 and two interior longitudinal beams 235. The linear guide assemblies 240 or any suitable slidable connectors may be provided at the four outer corners of the carriage frame 230 where the two exterior lateral beams 234 intersect the two outer longitudinal beams 235. The two interior main lateral beams 231 and two interior longitudinal beams 235 of the carriage frame 230 may support a swivel bearing housing 236 configured to house a swivel bearing 147 that pivotally couples the lower ceiling mount 146 to the upper ceiling mount 140 to allow the medical imaging system (e.g., X-ray imaging system 100) to rotate 360 degrees.

In a representative embodiment, the ends of each of the two interior main lateral beams 231 may comprise carriage safety hooks 170. For example, the two interior main lateral beams 231 may comprise an interior channel and the carriage safety hooks 170 may extend from the interior channel at both ends of each of the two interior main lateral beams 231. The carriage safety hooks 170 may be positioned to surround the protrusion 252 of the ceiling rails 250. In various embodiments, the carriage safety hooks 170 may not contact the protrusion 252 of the ceiling rails 250 and/or may not support a load of the medical imaging system (e.g., X-ray imaging system 100) in a normal condition (i.e., when a proper connection is present between the carriage frame 230 of the chassis 141 and the protrusion 252 of the ceiling rails 250 by the linear guide assemblies 240 and/or any suitable slidable connectors). Instead, the carriage safety hooks 170 may contact the protrusion 252 of the ceiling rail 250 to receive a load of the medical imaging system (e.g., X-ray imaging system 100) in a failure condition (i.e., when one or more of the linear guide assemblies 240 or any suitable slidable connectors coupling the carriage frame 230 of the chassis 141 to the protrusion 252 of the ceiling rails 250 fail).

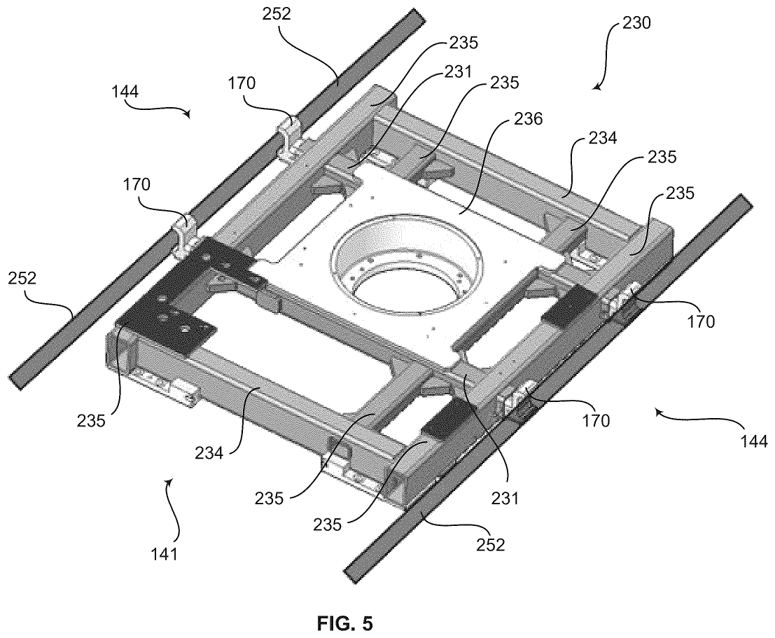

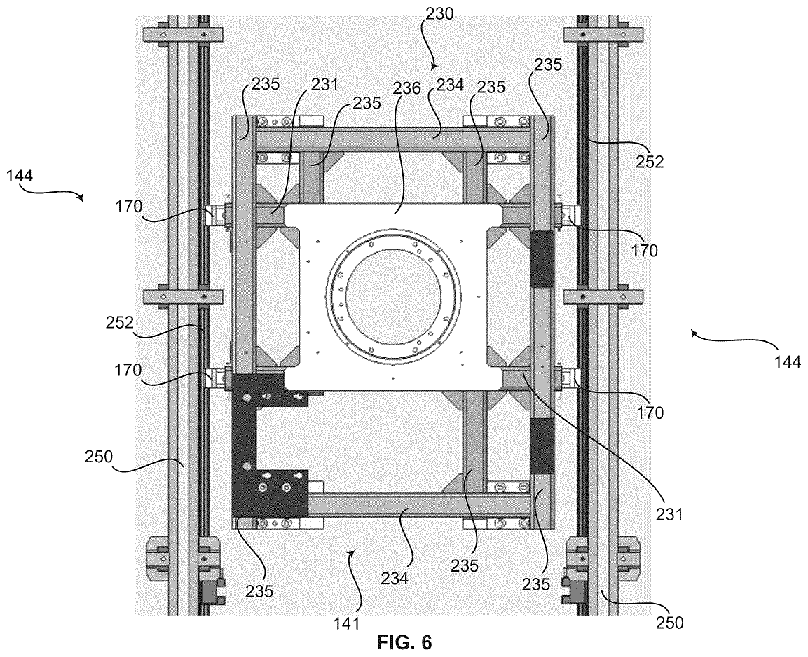

FIG. 5 is a schematic diagram of a perspective view of exemplary carriage safety hooks 170 of a carriage frame 230 of a chassis 141 of an upper ceiling mount 140 positioned to surround protrusions 252 of ceiling rails 250 of a rail system 144, in accordance with various embodiments. FIG. 6 is a schematic diagram of a top view of exemplary carriage safety hooks 170 of a carriage frame 230 of a chassis 141 of an upper ceiling mount 140 positioned to surround protrusions 252 of ceiling rails 250 of a rail system, in accordance with various embodiments. The chassis 141 having the carriage frame 230 configured for installation to a protrusion 252 of ceiling rails 250 of a rail system 144 as shown in FIGS. 5-6 shares various characteristics with the chassis 141 having the carriage frame 230 configured for installation to ceiling rails 250 of a rail system 144 as described above with reference to FIGS. 1-4. Various components, such as the ceiling rails and/or linear guides assemblies, among other things, may not be shown in FIGS. 5 and/or 6 to provide a clearer view of other components of the carriage frame 230 of the chassis 141.

Referring to FIGS. 5 and 6, a chassis 141 comprises a carriage frame 230 having lateral beams 231, 234 and longitudinal beams 235. The lateral beams 231, 234 include two outer lateral beams 234 and two interior main lateral beams 231. The longitudinal beams 235 include two outer longitudinal beams 235 and two interior longitudinal beams 235. The two interior main lateral beams 231 and two interior longitudinal beams 235 of the carriage frame 230 may support a swivel bearing housing 236 configured to house a swivel bearing that pivotally couples a lower ceiling mount to the chassis 141. In various embodiments, the ends of each of the two interior main lateral beams 231 comprise carriage safety hooks 170. For example, the two interior main lateral beams 231 may comprise an interior channel and the carriage safety hooks 170 may extend from the interior channel at both ends of each of the two interior main lateral beams 231. The carriage safety hooks 170 may be positioned to surround a protrusion 252 of ceiling rails 250 of a rail system 144 such that the carriage safety hooks 170 may catch the protrusion 252 of the ceiling rail 250 to prevent the carriage frame 230 from falling in a failure condition (i.e., when one or more of the linear guide assemblies or any suitable slidable connectors coupling the carriage frame 230 of the chassis 141 to the protrusion 252 of the ceiling rails 250 fail).

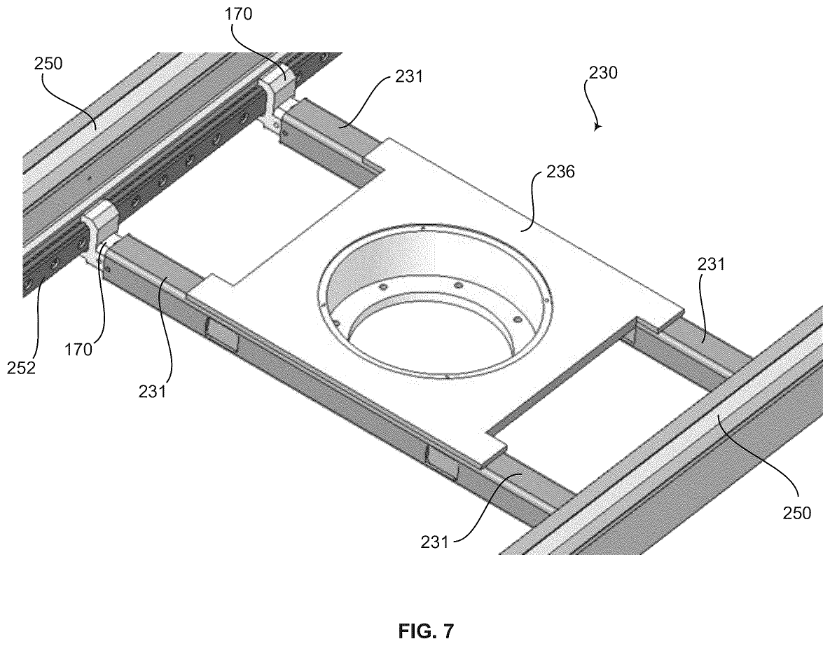

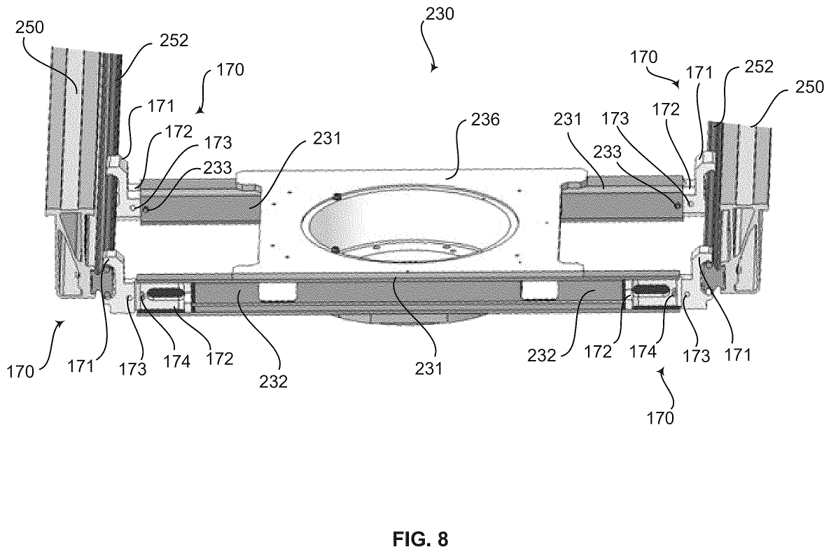

FIG. 7 is a schematic diagram of a top perspective view of exemplary carriage safety hooks 170 of a portion of a carriage frame 230 of a chassis 141 of an upper ceiling mount positioned to surround protrusions 252 of ceiling rails 250 of a rail system 144, in accordance with various embodiments. FIG. 8 is a schematic diagram of a side perspective view of exemplary carriage safety hooks 170 of a portion of a carriage frame 230 of a chassis 141 of an upper ceiling mount positioned to surround protrusions 252 of ceiling rails 250 of a rail system 144, in accordance with various embodiments. The chassis 141 having the carriage frame 230 with carriage safety hooks 170 as shown in FIGS. 7 and 8 share various characteristics with the chassis 141 having the carriage frame 230 with carriage safety hooks 170 as described above with reference to FIGS. 3-6. Various components, such as a portion of the carriage frame 230, may not be shown in FIGS. 7 and/or 8 to provide a clearer view of other components of the carriage frame 230, such as the carriage safety hooks 170.

Referring to FIGS. 7 and 8, the carriage frame 230 comprises two interior main lateral beams 231. The two interior main lateral beams 231 of the carriage frame 230 may support a swivel bearing housing 236 configured to house a swivel bearing that pivotally couples a lower ceiling mount to the carriage frame 230 of a chassis of an upper ceiling mount. In an exemplary embodiment, carriage safety hooks 170 extend from ends of each of the two interior main lateral beams 231. As shown in FIG. 8, for example, the interior main lateral beams 231 may comprise an interior channel 232. The carriage safety hooks 170 may comprise a safety hook 171 and a main body 172. The main body 172 of the carriage safety hook 170 may be inserted into the interior channel 232 of the interior main lateral beam 231. The main body 172 of the carriage safety hook 170 may comprise a first pin hole 173 and a second pin hole 174. The interior main lateral beams 231 may also comprise a pin hole 233 at each end of each interior main lateral beam 231. The carriage safety hook 170 may be secured at a retracted position by inserting a locking pin through the pin hole 233 in the interior main lateral beam 231 and the first pin hole 173 in the main body 172 of the carriage safety hook 170. The carriage safety hook 170 may be secured at an extended position, as shown in FIGS. 7 and 8, by inserting a locking pin through the pin hole 233 in the interior main lateral beam 231 and the second pin hole 174 in the main body 172 of the carriage safety hook 170.

The safety hook 171 of the carriage safety hook 170 may extend from the main body 172 of the carriage safety hook 170. The safety hook 171 may be generally C-shaped to partially surround a protrusion 252 on an interior side of the ceiling rail 250. In an exemplary embodiment, the carriage safety hook 170 comprising the safety hook 171 and the main body 172 is a single integrated piece that may be made of steel and/or any suitable high strength material. In various embodiments, the carriage safety hooks 170 may not contact the protrusion 252 of the ceiling rails 250 and/or may not support a load of the carriage frame 230 in a normal condition. Instead, the carriage safety hooks 170 may contact the protrusion 252 of the ceiling rail 250 to receive a load of the carriage frame 230 in a failure condition (i.e., when one or more of the attachment mechanisms slidably coupling the carriage frame 230 to the protrusion 252 of the ceiling rails 250 fail).

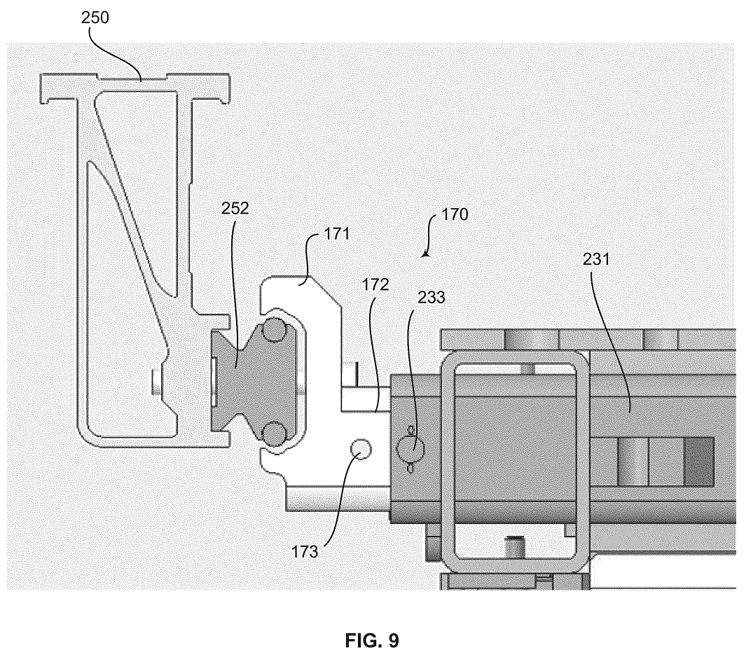

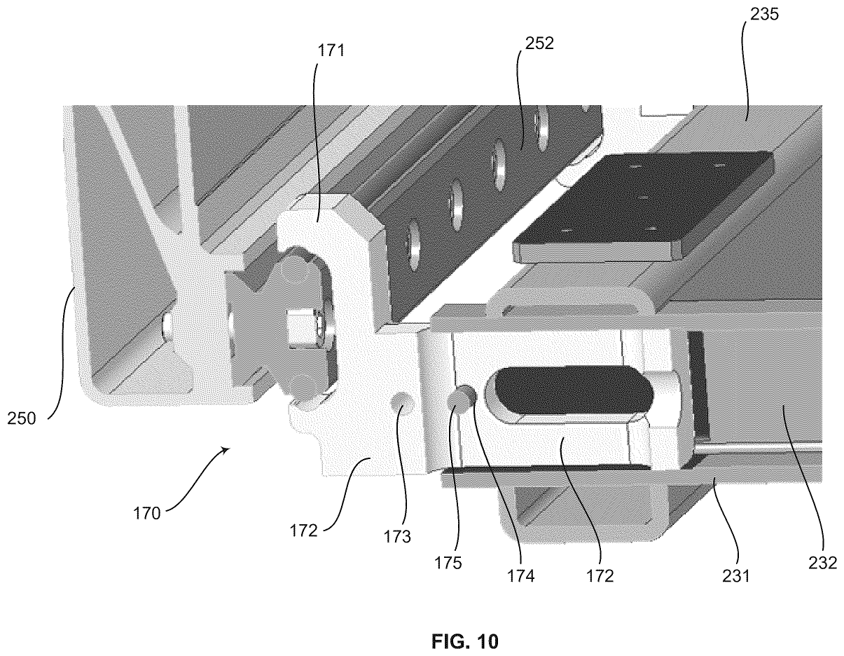

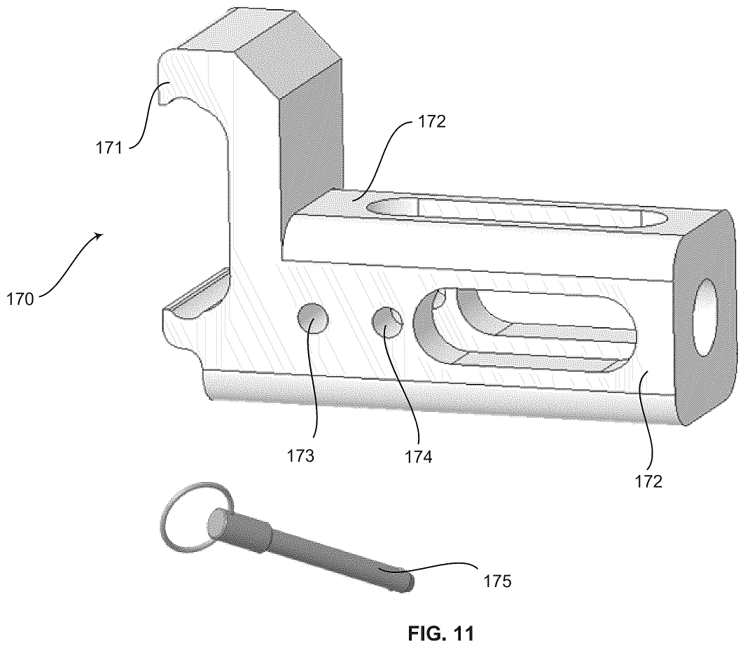

FIG. 9 is a schematic diagram of a lateral view of an exemplary carriage safety hook 170 extending from a main beam 231 of a carriage frame 230 and positioned to surround a protrusion 252 of a ceiling rail 250, in accordance with various embodiments. FIG. 10 is a schematic diagram of a cross-sectional view of an exemplary carriage safety hook 170 extending from a main beam 231 of a carriage frame 230 and positioned to surround a protrusion 252 of a ceiling rail 250, in accordance with various embodiments. FIG. 11 is a schematic diagram of a perspective view of an exemplary carriage safety hook 170 with locking pin 175, in accordance with various embodiments. The carriage safety hooks 170 as shown in FIGS. 9-11 share various characteristics with the carriage safety hooks 170 as described above with reference to FIGS. 3-8.

Referring to FIGS. 9-11, the carriage safety hook 170 comprises a safety hook 171 and a main body 172. The main body 172 of the carriage safety hook 170 may be inserted into an interior channel 232 of an interior main lateral beam 231 of a carriage frame. The main body 172 of the carriage safety hook 170 may comprise a first pin hole 173 and a second pin hole 174. The interior main lateral beams 231 may also comprise a pin hole 233 at each end of each interior main lateral beam 231. The carriage safety hook 170 may be secured at a retracted position by inserting a locking pin through the pin hole 233 in the interior main lateral beam 231 and the first pin hole 173 in the main body 172 of the carriage safety hook 170. The carriage safety hook 170 may be secured at an extended position, as shown in FIGS. 9 and 10, by inserting a locking pin 175 through the pin hole 233 in the interior main lateral beam 231 and the second pin hole 174 in the main body 172 of the carriage safety hook 170. The locking pin 175 may be a headed quick release ball lock pin with ring or any suitable locking pin. The safety hook 171 of the carriage safety hook 170 may extend from the main body 172 of the carriage safety hook 170. The safety hook 171 may be generally C-shaped to partially surround a protrusion 252 on an interior side of a ceiling rail 250. In an exemplary embodiment, the carriage safety hook 170 comprising the safety hook 171 and the main body 172 is a single integrated piece that may be made of steel and/or any suitable high strength material. In certain embodiments, interior portions of the main body 232 of the carriage safety hooks 170 may be removed to reduce the weight and/or cost of the carriage safety hooks 170. In various embodiments, the carriage safety hooks 170 may not contact the protrusion 252 of the ceiling rails 250 and/or may not support a load of the carriage frame in a normal condition. Instead, the carriage safety hooks 170 may contact the protrusion 252 of the ceiling rail 250 to receive a load of the carriage frame 230 in a failure condition (i.e., when one or more of the attachment mechanisms slidably coupling the carriage frame to the protrusion 252 of the ceiling rails 250 fail).

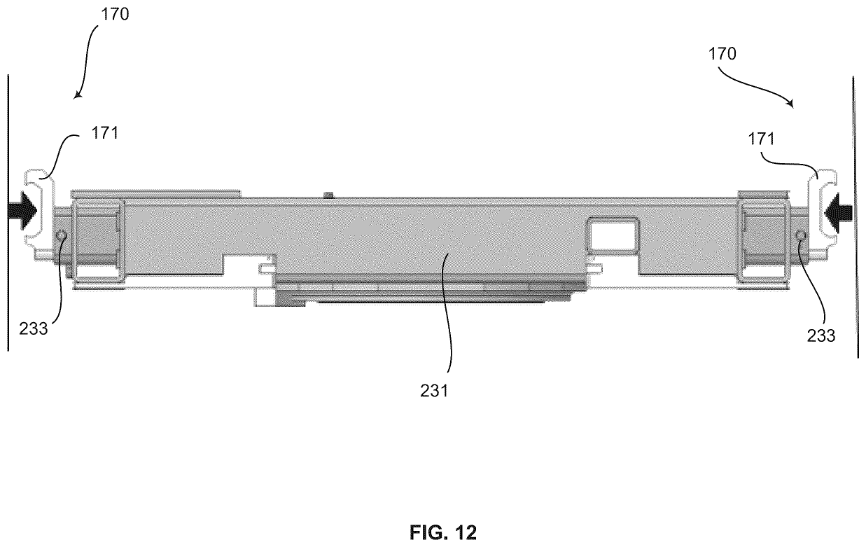

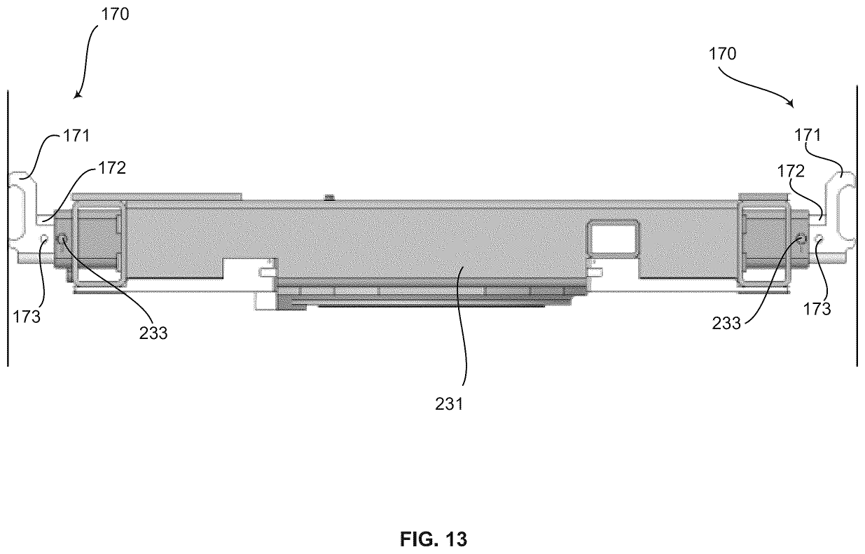

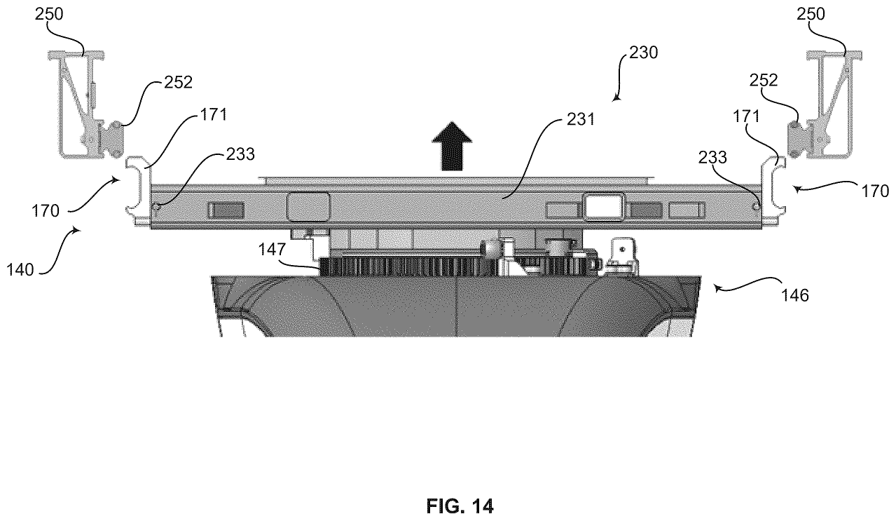

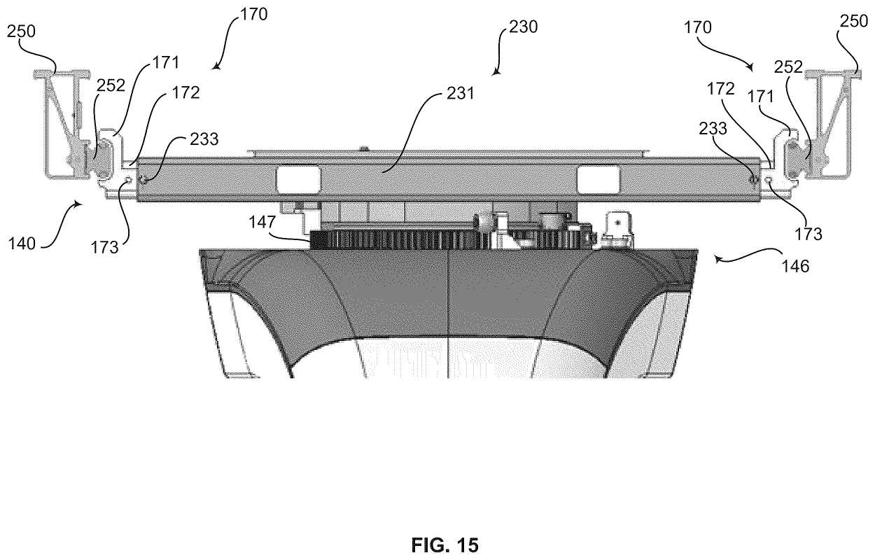

FIG. 12 is a schematic diagram of a lateral view of a main beam 231 of a carriage frame of a chassis having carriage safety hooks 170 provided in a retracted position, in accordance with various embodiments. FIG. 13 is a schematic diagram of a lateral view of a main beam 231 of a carriage frame of a chassis having carriage safety hooks 170 provided in an extended position, in accordance with various embodiments. FIG. 14 is a schematic diagram of a lateral view of an exemplary carriage frame 230 of a chassis of an upper ceiling mount 140 pivotally coupled to a lower ceiling mount 146 by a swivel bearing 147, the carriage frame 230 having carriage safety hooks 170 provided in a retracted position prior to installation on protrusions 252 of ceiling rails 250, in accordance with various embodiments. FIG. 15 is a schematic diagram of a lateral view of an exemplary carriage frame 230 of a chassis of an upper ceiling mount 140 pivotally coupled to a lower ceiling mount 146 by a swivel bearing 147, the carriage frame 230 having carriage safety hooks 170 provided in an extended position after installation on protrusions 252 of ceiling rails 250, in accordance with various embodiments. The carriage safety hooks 170 shown in FIGS. 12-15 share various characteristics with the carriage safety hooks 170 as described above with reference to FIGS. 3-11.

Referring to FIGS. 12-15, the carriage safety hook 170 comprises a safety hook 171 and a main body 172. The main body 172 of the carriage safety hook 170 may be inserted into an interior channel of an interior main lateral beam 231 of a carriage frame. The main body 172 of the carriage safety hook 170 may comprise a first pin hole 173 and a second pin hole. The interior main lateral beams 231 may also comprise a pin hole 233 at each end of each interior main lateral beam 231. The carriage safety hook 170 may be secured at a retracted position, as shown in FIGS. 12 and 14, by inserting a locking pin through the pin hole 233 in the interior main lateral beam 231 and the first pin hole 173 in the main body 172 of the carriage safety hook 170. The carriage safety hook 170 may be secured at an extended position, as shown in FIGS. 13 and 15, by inserting a locking pin through the pin hole 233 in the interior main lateral beam 231 and the second pin hole in the main body 172 of the carriage safety hook 170. The locking pin 175 may be a headed quick release ball lock pin with ring or any suitable locking pin. The safety hook 171 of the carriage safety hook 170 may extend from the main body 172 of the carriage safety hook 170. The safety hook 171 may be generally C-shaped to partially surround a protrusion 252 on an interior side of a ceiling rail 250. In an exemplary embodiment, the carriage safety hook 170 comprising the safety hook 171 and the main body 172 is a single integrated piece that may be made of steel and/or any suitable high strength material.

As shown in FIGS. 14 and 15, the carriage frame 230 of an upper ceiling mount 140 may be pivotally coupled to a lower ceiling mount 146 by a swivel bearing 147. A medical imaging system, such as an X-ray imaging system 100 or any suitable medical imaging system, may be coupled to the lower ceiling mount 146. To install the medical imaging system to the ceiling with the upper 140 and lower 146 ceiling mounts, the carriage frame 230 of the upper ceiling mount 140 is slidably coupled to ceiling rails 250 of the rail system 144 of the upper ceiling mount 140. As shown in FIG. 14, prior to lifting the carriage frame 230 to slidably couple the carriage frame 230 to the ceiling rails 250 by linear guide assemblies or any suitable slidable connectors, the carriage safety hooks 170 may be provided in a retracted position so the carriage safety hooks 170 do not interfere with the mounting of the carriage frame 230 on protrusions 252 of ceiling rails 250. As shown in FIG. 15, after the carriage frame 230 is slidably coupled to the protrusions 252 of the ceiling rails 250, the carriage safety hooks 170 may be extended so that the safety hook 171 of the carriage safety hooks 170 wrap partially around the protrusions 252 of the ceiling rails 250. The carriage safety hooks 170 may be extended by removing a locking pin inserted through the pin hole 233 of the interior main lateral beam 231 and the first pin hole 173 of the main body 172 of the carriage safety hook 170. A portion of the main body 172 of the carriage safety hook 170 may then be pulled out of an interior channel 232 of the interior main lateral beam 231 until the second pin hole of the carriage safety hook 170 aligns with the pin hole 233 of the interior main lateral beam 231. The locking pin is then inserted through the pin hole 233 of the interior main lateral beam 231 and the second pin hole of the main body 172 of the carriage safety hook 170 to secure the carriage safety hook 170 in the extended position.

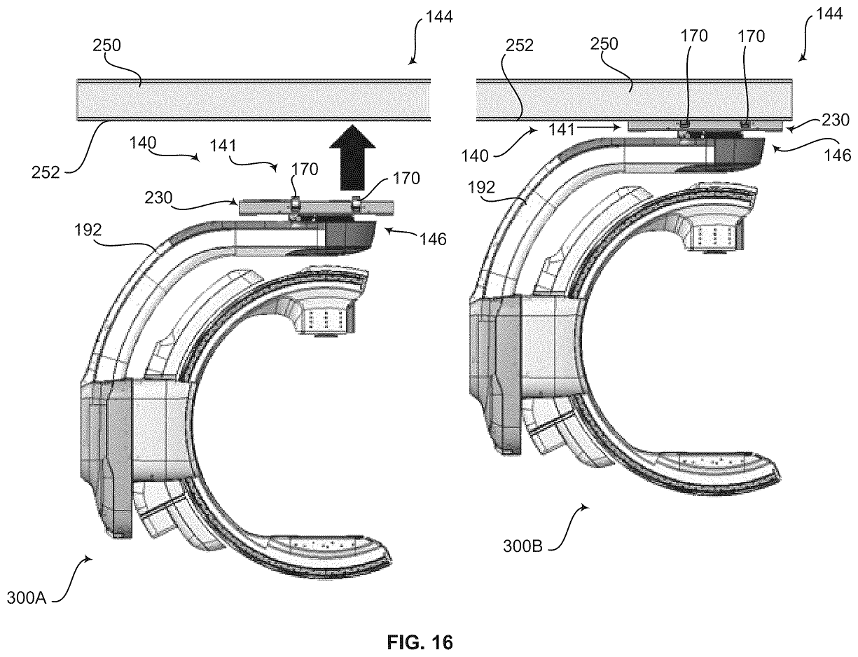

FIG. 16 is a schematic diagram of lateral views of a preferred installation method of a medical imaging system 300A, 300B to ceiling rails 250 of a rail system 144 by a ceiling mount assembly 140, 146 having carriage safety hooks 170, in accordance with various embodiments. The ceiling mount assembly 140, 146 having carriage safety hooks 170 shown in FIG. 16 shares various characteristics with the ceiling mount assembly 140, 146 having carriage safety hooks 170 as described above with reference to FIGS. 1-15. Referring to FIG. 16, a medical imaging system is shown prior to installation 300A and after installation 300B. The medical imaging system 300A, 300B is being installed on protrusions 252 of ceiling rails 250. The upper ceiling mount comprises the rail system 144 having the ceiling rails 250 and a chassis 141 having a carriage frame 230 with carriage safety hooks 170. A mounting structure 192 (e.g., an L-arm) of the medical imaging system (e.g., an X-ray imaging system) is coupled to a lower ceiling mount 146, which is pivotally coupled to the upper ceiling mount 140. In the embodiment of FIG. 16, the medical imaging system 300A, 300B is installed by slidably coupling the carriage frame 230 of the chassis 141 to the protrusions 252 of the ceiling rails 250 of the rail system 144. The medical imaging system 300A, 300B may be slidably coupled to the protrusions 252 of the ceiling rails 250 by linear guide assemblies, or any suitable slidable connectors.

As discussed above with respect to FIGS. 14 and 15, the carriage safety hooks 170 may be provided in a retracted position prior to installation 300A so the carriage safety hooks 170 do not interfere with the mounting of the carriage frame 230 on protrusions 252 of ceiling rails 250. After the carriage frame 230 is slidably coupled to the protrusions 252 of the ceiling rails 250, the carriage safety hooks 170 may be extended so that the safety hook of the carriage safety hooks 170 wrap partially around the protrusions 252 of the ceiling rails 250. The extendable and retractable carriage safety hooks 170 allow the ceiling mount assembly 140, 146 to be lifted vertically to install the medical imaging system 300A, 300B to the ceiling rails 250 of the rail system 144 instead of having to slide the carriage frame 230 with extended carriage safety hooks 170 horizontally over the protrusions 252 of the ceiling rails 250 from an end of the ceiling rails 250. Accordingly, the installation method of FIG. 16 allows installation of the medical imaging system 300A, 300B to ceilings of smaller examination rooms and/or allows longer ceiling rails 250 for greater lateral movement within an examination room.

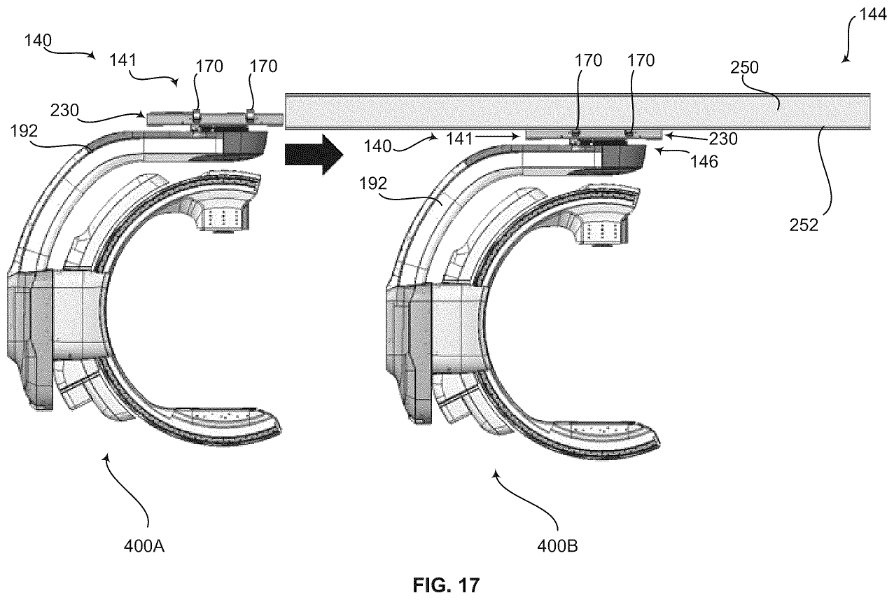

FIG. 17 is a schematic diagram of lateral views of an alternative installation method of a medical imaging system 400A, 400B to ceiling rails 250 of a rail system 144 by a ceiling mount assembly 140, 146 having carriage safety hooks 170, in accordance with various embodiments. The ceiling mount assembly 140, 146 having carriage safety hooks 170 shown in FIG. 17 shares various characteristics with the ceiling mount assembly 140, 146 having carriage safety hooks 170 as described above with reference to FIGS. 1-16. Referring to FIG. 17, a medical imaging system is shown prior to installation 400A and after installation 400B. The medical imaging system 400A, 400B is being installed on protrusions 252 of ceiling rails 250. The upper ceiling mount comprises the rail system 144 having the ceiling rails 250 and a chassis 141 having a carriage frame 230 with carriage safety hooks 170. A mounting structure 192 (e.g., an L-arm) of the medical imaging system (e.g., an X-ray imaging system) is coupled to a lower ceiling mount 146, which is pivotally coupled to the upper ceiling mount 140. In the embodiment of FIG. 17, the medical imaging system 400A, 400B is installed by sliding the carriage frame 230 with extended carriage safety hooks 170 horizontally over the protrusions 252 of the ceiling rails 250 from an end of the ceiling rails 250. The method of installation of the medical imaging system 400A, 400B of FIG. 17 may require a larger examination room and/or shorter ceiling rails 250 to provide enough space between the end of the ceiling rails 250 and walls of the examination room to allow the medical imaging system 400A, 400B to be slid horizontally over an end of the ceiling rails 250. In the embodiment of FIG. 17, the carriage safety hooks 170 may be left in an extended position instead of having to retract the carriage safety hooks 170 prior to installation and extending the carriage safety hooks 170 after installation as shown in FIG. 16.

Although various embodiments described above provide an X-ray imaging system 100 mounted to a ceiling 180 by and upper ceiling mount 140 and a lower ceiling mount 146 where the upper ceiling mount comprises carriage safety hooks 170, unless so claimed the carriage safety hooks 170 are not limited to use in ceiling mounts 140, 146 for X-ray imaging systems 100, and may be provided in ceiling mounts 140, 146 of any suitable medical imaging systems.

Aspects of the present disclosure provide a carriage safety hook 170 of a medical imaging system ceiling mount assembly 140, 146 operable to receive a load of a medical imaging system 100 in a failure condition of the medical imaging system ceiling mount assembly 140, 146. The medical imaging system ceiling mount assembly 140, 146 may comprise ceiling rails 250 and a carriage frame 230 coupled to the medical imaging system 100. The carriage safety hook 170 may comprise a main body 172, a hook 171, and a locking pin 175. The main body 172 may comprise a first end and a second end. The first end may be slidably insertable in an interior channel 232 of a main beam 231 of the carriage frame 230. The hook 171 may extend from the second end of the main body 172 and configured to partially surround a protrusion 252 of the one of the ceiling rails 250. The main body 172 may comprise a first pin hole 173 extending through the main body 172. The main body 172 may comprise a second pin hole 174 extending through the main body 172. The second pin hole 174 may be positioned between the first pin hole 173 and the first end of the main body 172. The first pin hole 173 may be positioned between the second pin hole 174 and the second end of the main body 172. The locking pin 175 may be inserted through an aperture 233 in the main beam 231 of the carriage frame 230 and the first pin hole 173 to secure the carriage safety hook 170 in a retracted position. The locking pin 175 may be inserted through the aperture 233 in the main beam 231 of the carriage frame 230 and the second pin hole 174 to secure the carriage safety hook 170 in an extended position.

In an exemplary embodiment, the carriage frame 230 may comprise slidable connectors 240 and a plurality of the carriage safety hook 170. Each of the slidable connectors 240 may be configured to slidably couple with a protrusion 252 of one of the ceiling rails 250 in a normal condition. Each of the plurality of the carriage safety hook 170 may be configured to receive the protrusion 252 of the one of the ceiling rails 250 in the failure condition. The normal condition corresponds with normal connections between the carriage frame 230 and the protrusion 252 of the one of the ceiling rails 250 by each of the slidable connectors 240. The failure condition corresponds with a failed connection between the carriage frame 230 and the protrusion 252 of the one of the ceiling rails 250 by one or more of the slidable connectors 240. In a representative embodiment, the carriage safety hook 170 does not receive the load of the medical imaging system 100 in the normal condition. In various embodiments, the carriage safety hook 170 does not contact the protrusion 252 of the one of the ceiling rails 250 in the normal condition. In certain embodiments, the locking pin 175 is a headed quick release ball lock pin with ring. In an exemplary embodiment, the hook 171 is C-shaped. In a representative embodiment, the carriage safety hook 170 comprises steel. In certain embodiments, an interior portion of the main body 172 between the first end and the second end of the main body 172 is void of material.

Various embodiments provide ceiling mount assembly 140, 146 operable to mount a medical imaging system 100 to a ceiling 180. The ceiling mount assembly 140, 146 may comprise two parallel ceiling rails 250 and a carriage frame 230. The two parallel ceiling rails 250 may be coupled to the ceiling 180. Each of the two parallel ceiling rails 250 may comprise an exterior side facing away from an other of the two parallel ceiling rails 250 and an interior side facing the other of the two parallel ceiling rails 250. The interior side of each of the two parallel ceiling rails 250 may comprise a protrusion 252. The carriage frame 230 may be coupled to the medical imaging system 100. The carriage frame 230 may comprise outer beams 234, 235 and interior main beams 231. The carriage frame 230 may comprise slidable connectors 240 on the outer beams 234, 235 and carriage safety hooks 170 extending from interior channels 232 of the interior main beams 231. Each of the slidable connectors 240 may be configured to slidably couple with the protrusion 252 of one of the two parallel ceiling rails 250 in a normal condition. Each of the plurality of the carriage safety hooks 170 may be configured to receive the protrusion 252 of the one of the two parallel ceiling rails 250 in a failure condition of the ceiling mount assembly 140, 146. Each of the plurality of carriage safety hooks 170 may comprise a main body 172, a hook 171, and a locking pin 175. The main body 172 may comprise a first end and a second end. The first end may be slidably insertable in one of the interior channels 232 of one of the interior main beams 231 of the carriage frame 230. The hook 171 may extend from the second end of the main body 172 and configured to partially surround the protrusion 252 of the one of the two parallel ceiling rails 250. The main body 172 may comprise a first pin hole 173 extending through the main body 172. The main body 172 may comprise a second pin hole 174 extending through the main body 172. The second pin hole 174 may be positioned between the first pin hole 173 and the first end of the main body 172. The first pin hole 173 may be positioned between the second pin hole 174 and the second end of the main body 172. The locking pin 175 may be inserted through an aperture 233 in the one of the interior main beams 231 of the carriage frame 230 and the first pin hole 173 to secure a corresponding one of the plurality of carriage safety hooks 170 in a retracted position. The locking pin 175 may be inserted through the aperture 233 in the one of the interior main beams 231 of the carriage frame 230 and the second pin hole 174 to secure the corresponding one of the plurality of carriage safety hooks 170 in an extended position.

In a representative embodiment, the normal condition corresponds with normal connections between the carriage frame 230 and the protrusion 252 of the one of the ceiling rails 250 by each of the slidable connectors 240. The failure condition corresponds with a failed connection between the carriage frame 230 and the protrusion 252 of the one of the ceiling rails 250 by one or more of the slidable connectors 240. In various embodiments, each of the plurality of carriage safety hooks 170 does not receive a load of the medical imaging system 100 in the normal condition. In certain embodiments, the locking pin 175 is a headed quick release ball lock pin with ring 175. In an exemplary embodiment, the hook 171 is C-shaped. In a representative embodiment, each of the plurality of carriage safety hooks 170 comprises steel. In various embodiments, the slidable connectors 240 comprise linear guide assemblies 240 having rollers.

Certain embodiments provide a ceiling mounted medical imaging system 100 comprising a medical imaging system 100 operable to acquire medical image data, and a ceiling mount assembly 140, 146. The ceiling mount assembly 140, 146 may comprise two parallel ceiling rails 250 and a carriage frame 230. The two parallel ceiling rails 250 may be coupled to the ceiling 180. Each of the two parallel ceiling rails 250 may comprise an exterior side facing away from an other of the two parallel ceiling rails 250 and an interior side facing the other of the two parallel ceiling rails 250. The interior side of each of the two parallel ceiling rails comprises a protrusion 252. The carriage frame 230 may be coupled to the medical imaging system 100. The carriage frame 230 may comprise outer beams 234, 235 and interior main beams 231. The carriage frame 230 may comprise slidable connectors 240 on the outer beams 234, 235 and carriage safety hooks 170 extending from interior channels 232 of the interior main beams 231. Each of the slidable connectors 240 may be configured to slidably couple with the protrusion 252 of one of the two parallel ceiling rails 250 in a normal condition. Each of the plurality of the carriage safety hooks 170 may be configured to receive the protrusion 252 of the one of the two parallel ceiling rails 250 in a failure condition of the ceiling mount assembly 140, 146. The failure condition may correspond with a failed connection between the carriage frame 230 and the protrusion 252 of the one of the ceiling rails 250 by one or more of the slidable connectors 240. Each of the plurality of carriage safety hooks 170 may comprise a main body 172, a C-shaped hook 171, and a locking pin 175. The main body 172 may comprise a first end and a second end. The first end may be slidably insertable in one of the interior channels 232 of one of the interior main beams 231 of the carriage frame 230. The C-shaped hook 171 may extend from the second end of the main body 172 and configured to partially surround the protrusion 252 of the one of the two parallel ceiling rails 250. The main body 172 may comprise a first pin hole 173 extending through the main body 172. The main body 172 may comprise a second pin hole 174 extending through the main body 172. The second pin hole 174 may be positioned between the first pin hole 173 and the first end of the main body 172. The first pin hole 173 may be positioned between the second pin hole 174 and the second end of the main body 172. The locking pin 175 may be inserted through an aperture 233 in the one of the interior main beams 231 of the carriage frame 230 and the first pin hole 173 to secure a corresponding one of the plurality of carriage safety hooks 170 in a retracted position. The locking pin 175 may be inserted through the aperture 233 in the one of the interior main beams 231 of the carriage frame 230 and the second pin hole 174 to secure the corresponding one of the plurality of carriage safety hooks 170 in an extended position.

In various embodiments, the ceiling mounted medical imaging system 100 is an X-ray medical imaging system 100. In certain embodiments, each of the plurality of carriage safety hooks 170 does not receive a load of the medical imaging system 100 in the normal condition. In an exemplary embodiment, at least one of the plurality of carriage safety hooks 170 receives a load of the medical imaging system 100 in the failure condition. In a representative embodiment, the locking pin 175 secures each of the plurality of carriage safety hooks 170 in the retracted position prior to slidably coupling each of the slidable connectors 240 with the protrusion 252 of one of the two parallel ceiling rails 250 during installation of the ceiling mounted medical imaging system. The locking pin 175 secures each of the plurality of carriage safety hooks 170 in the extended position after all of the slidable connectors 240 are slidably coupled with the protrusion 252 of the two parallel ceiling rails 250.

As utilized herein the term “circuitry” refers to physical electronic components (i.e., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, a component is “operable” or “configured” to perform a function whenever the component comprises the necessary structure to perform the function, regardless of whether the function is performed.

While the present disclosure has been described with reference to certain embodiments, it will be understood by those skilled in the art that various changes may be made, and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A carriage safety hook of a medical imaging system ceiling mount assembly operable to receive a load of a medical imaging system in a failure condition of the medical imaging system ceiling mount assembly, the medical imaging system ceiling mount assembly comprising ceiling rails and a carriage frame coupled to the medical imaging system, the carriage safety hook comprising:
a main body having a first end and a second end, the first end slidably insertable in an interior channel of a main beam of the carriage frame;
a hook extending from the second end of the main body and configured to partially surround a protrusion of the one of the ceiling rails; and
a locking pin,
wherein:
the main body comprises:
a first pin hole extending through the main body; and
a second pin hole extending through the main body, the second pin hole positioned between the first pin hole and the first end of the main body, and the first pin hole positioned between the second pin hole and the second end of the main body;
the locking pin is inserted through an aperture in the main beam of the carriage frame and the first pin hole to secure the carriage safety hook in a retracted position; and
the locking pin is inserted through the aperture in the main beam of the carriage frame and the second pin hole to secure the carriage safety hook in an extended position.

2. The carriage safety hook of claim 1, wherein:
the carriage frame having slidable connectors and a plurality of the carriage safety hook, each of the slidable connectors configured to slidably couple with a protrusion of one of the ceiling rails in a normal condition, and each of the plurality of the carriage safety hook configured to receive the protrusion of the one of the ceiling rails in the failure condition;
the normal condition corresponds with normal connections between the carriage frame and the protrusion of the one of the ceiling rails by each of the slidable connectors; and
the failure condition corresponds with a failed connection between the carriage frame and the protrusion of the one of the ceiling rails by one or more of the slidable connectors.

3. The carriage safety hook of claim 2, wherein the carriage safety hook does not receive the load of the medical imaging system in the normal condition.

4. The carriage safety hook of claim 2, wherein the carriage safety hook does not contact the protrusion of the one of the ceiling rails in the normal condition.

5. The carriage safety hook of claim 1, wherein the locking pin is a headed quick release ball lock pin with ring.

6. The carriage safety hook of claim 1, wherein the hook is C-shaped.

7. The carriage safety hook of claim 1, wherein the carriage safety hook comprises steel.

8. The carriage safety hook of claim 1, wherein an interior portion of the main body between the first end and the second end of the main body is void of material.

9. A ceiling mount assembly operable to mount a medical imaging system to a ceiling, the ceiling mount assembly comprising:
two parallel ceiling rails coupled to the ceiling, each of the two parallel ceiling rails comprising an exterior side facing away from an other of the two parallel ceiling rails and an interior side facing the other of the two parallel ceiling rails, wherein the interior side of each of the two parallel ceiling rails comprises a protrusion; and
a carriage frame coupled to the medical imaging system, the carriage frame having outer beams and interior main beams, the carriage frame comprising slidable connectors on the outer beams and carriage safety hooks extending from interior channels of the interior main beams, each of the slidable connectors configured to slidably couple with the protrusion of one of the two parallel ceiling rails in a normal condition, and each of the plurality of the carriage safety hooks configured to receive the protrusion of the one of the two parallel ceiling rails in a failure condition of the ceiling mount assembly, wherein each of the plurality of carriage safety hooks comprises:
a main body having a first end and a second end, the first end slidably insertable in one of the interior channels of one of the interior main beams of the carriage frame;
a hook extending from the second end of the main body and configured to partially surround the protrusion of the one of the two parallel ceiling rails; and
a locking pin,
wherein:
the main body comprises:
a first pin hole extending through the main body; and
a second pin hole extending through the main body, the second pin hole positioned between the first pin hole and the first end of the main body, and the first pin hole positioned between the second pin hole and the second end of the main body;
the locking pin is inserted through an aperture in the one of the interior main beams of the carriage frame and the first pin hole to secure a corresponding one of the plurality of carriage safety hooks in a retracted position; and
the locking pin is inserted through the aperture in the one of the interior main beams of the carriage frame and the second pin hole to secure the corresponding one of the plurality of carriage safety hooks in an extended position.

10. The ceiling mount assembly of claim 9, wherein:
the normal condition corresponds with normal connections between the carriage frame and the protrusion of the one of the ceiling rails by each of the slidable connectors; and
the failure condition corresponds with a failed connection between the carriage frame and the protrusion of the one of the ceiling rails by one or more of the slidable connectors.

11. The ceiling mount assembly of claim 10, wherein each of the plurality of carriage safety hooks does not receive a load of the medical imaging system in the normal condition.

12. The ceiling mount assembly of claim 9, wherein the locking pin is a headed quick release ball lock pin with ring.

13. The ceiling mount assembly of claim 9, wherein the hook is C-shaped.

14. The ceiling mount assembly of claim 9, wherein each of the plurality of carriage safety hooks comprises steel.

15. The ceiling mount assembly of claim 9, wherein the slidable connectors comprise linear guide assemblies having rollers.

16. A ceiling mounted medical imaging system, comprising:
a medical imaging system operable to acquire medical image data; and
a ceiling mount assembly comprising:
two parallel ceiling rails coupled to the ceiling, each of the two parallel ceiling rails comprising an exterior side facing away from an other of the two parallel ceiling rails and an interior side facing the other of the two parallel ceiling rails, wherein the interior side of each of the two parallel ceiling rails comprises a protrusion; and
a carriage frame coupled to the medical imaging system, the carriage frame having outer beams and interior main beams, the carriage frame comprising slidable connectors on the outer beams and carriage safety hooks extending from interior channels of the interior main beams, each of the slidable connectors configured to slidably couple with the protrusion of one of the two parallel ceiling rails in a normal condition, and each of the plurality of the carriage safety hooks configured to receive the protrusion of the one of the two parallel ceiling rails in a failure condition of the ceiling mount assembly, wherein the failure condition corresponds with a failed connection between the carriage frame and the protrusion of the one of the ceiling rails by one or more of the slidable connectors, and wherein each of the plurality of carriage safety hooks comprises:
a main body having a first end and a second end, the first end slidably insertable in one of the interior channels of one of the interior main beams of the carriage frame;
a C-shaped hook extending from the second end of the main body and configured to partially surround the protrusion of the one of the two parallel ceiling rails; and
a locking pin,
wherein:
the main body comprises:
a first pin hole extending through the main body; and
a second pin hole extending through the main body,
the second pin hole positioned between the first pin hole and the first end of the main body, and the first pin hole positioned between the second pin hole and the second end of the main body;
the locking pin is inserted through an aperture in the one of the interior main beams of the carriage frame and the first pin hole to secure a corresponding one of the plurality of carriage safety hooks in a retracted position;
the locking pin is inserted through the aperture in the one of the interior main beams of the carriage frame and the second pin hole to secure the corresponding one of the plurality of carriage safety hooks in an extended position.

17. The ceiling mounted medical imaging system of claim 16, wherein the ceiling mounted medical imaging system is an X-ray medical imaging system.

18. The ceiling mounted medical imaging system of claim 16, wherein each of the plurality of carriage safety hooks does not receive a load of the medical imaging system in the normal condition.

19. The ceiling mounted medical imaging system of claim 16, wherein at least one of the plurality of carriage safety hooks receives a load of the medical imaging system in the failure condition.

20. The ceiling mounted medical imaging system of claim 16, wherein:
the locking pin secures each of the plurality of carriage safety hooks in the retracted position prior to slidably coupling each of the slidable connectors with the protrusion of one of the two parallel ceiling rails during installation of the ceiling mounted medical imaging system; and
the locking pin secures each of the plurality of carriage safety hooks in the extended position after all of the slidable connectors are slidably coupled with the protrusion of the two parallel ceiling rails.
